(19) United States
(12) Patent Application Publication
(10) Pub. No.: US 2006/0127315 A1
(43) Pub. Date: Jun. 15, 2006

(54) Methods of treating humans undergoing injection of a contrast agent

(75) Inventor: Sudhir V. Shah, Little Rock, AR (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: SHIVA BIOMEDICAL, L.L.C., PARAMUS, NJ (US)

(21) Appl. No.: 11/198,931

(22) Filed: Aug. 5, 2005

Related U.S. Application Data
(60) Provisional application No. 60/599,449, filed on Aug. 6, 2004.

(51) Int. Cl. A61K 49/00; A61K 31/195

(52) U.S. Cl. 424/9.3; 514/564; 514/566

(57) ABSTRACT

A human undergoing an injection of a contrast agent is treated by the administration of an iron chelator. Administration of the iron chelator can essentially prevent the onset of acute renal failure in a human. Administration of the iron chelator can reduce the severity of the acute renal failure and/or reduce the severity of kidney disease consequent to the acute renal failure.

### RELATED APPLICATION

This application claims the benefit of U.S. Provisional Application No. 60/599,449, filed Aug. 6, 2004, the entire teachings of the which are incorporated herein by reference.

### BACKGROUND OF THE INVENTION

Acute renal failure (ARF) is a sudden loss of the ability of the kidneys to excrete wastes, concentrate urine and conserve electrolytes. ARF is associated with a rapid decline in glomerular filtration rate (GFR) and retention of nitrogenous waste products in the blood (e.g., blood urea nitrogen). ARF can occur consequent to injection of a contrast agent. ARF has been reported in 5% of patients requiring hospitalizations and 30% of intense care unit admissions (Hou, S. H., et al., Am J. Med 74:243-248 (1983)). Expenses related to caring for patients with ARF are estimated at $8 billion per year. ARF has been associated with increased morbidity and mortality, cost of care and prolonged hospitalizations (Levy, E. M., et al., J Am Med Assoc 275: 1516-1517 (1996); Gruberg, L., et al., J Am College Cardiol 36: 1542-1548 (2000)). Currently available treatment options for ARF include the administration of dopamine, diuretics, calcium-channel antagonists, aminophylline, atrial natriuretic peptide, insulin-derived growth factor, acetylcysteine and hydration. However, such treatments do not alleviate the ARF, prevent progression of ARF or prevent irreversible kidney damage consequent to injection of a contrast agent. Thus, there is a need to develop new, improved and effective methods of treatment of a human undergoing injection of a contrast agent, in particular to treat ARF consequent to injection of the contrast agent.

### SUMMARY OF THE INVENTION

The present invention relates to a method of treating a human undergoing an injection of a contrast agent. The invention is a method of treating a human comprising the step of administering a iron chelator to a human undergoing an injection of a contrast agent.

The invention described herein provides methods for treating a human undergoing injection of a contrast agent. In a particular embodiment, administration of the iron chelator to the human undergoing an injection of a contrast agent essentially prevents the onset of acute renal failure in a human, reduces the severity of acute renal failure in the human or prevents irreversible kidney damage consequent to the injection of the contrast agent. Thus, advantages of the claimed invention can include, for example, treatment of acute renal failure consequent to injection of a contrast agent in a human in a manner that alleviates the acute renal failure, progression of the acute renal failure or irreversible changes to the kidneys. The methods of the invention can treat a human without significant side effects. The methods of the invention provide an effective manner to treat a human undergoing an injection of a contrast agent to thereby increase the quality of life and life expectancy of the human.

### BRIEF DESCRIPTION OF THE FIGURES

FIG. 1 depicts the steady state plasma levels of deferiprone concentration in human every 24 hours following the administration of an immediate release, a delayed release or an extended release formulation of deferiprone.

FIG. 2 depicts the steady state plasma levels of deferiprone concentration in human every 12 hours following the administration of an immediate release, a delayed release or an extended release formulation of deferiprone.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention is a method of treating a human, comprising the step of administering an iron chelator to human undergoing an injection of a contrast agent. A “contrast agent,” as used herein, refers to a compound employed to improve the visibility of internal body structures in an image, such as an X-ray image or a scanning image (e.g., CAT (Computerized Axial Tomography) scan, MRI (Magnetic Resonance Imaging) scan). The term contrast agent is also referred to herein as a radiocontrast agent. Contrast agents are employed in various diagnostic (e.g., cardiac catheterization) and therapeutic (e.g., vascular shunt placement) procedures.

In one embodiment, the contrast agent can be an ionic contrast agent. In another embodiment, the contrast agent can be a non-ionic contrast agent.

Ionic contrast agent suitable for use in the methods of the invention include Renovue®-DIP (iodamide meglumine) (Squibb), Conray® 30 (iothalamate meglumine) (Mallinckrodt), Hypaque® Meglumine 30% (diatrizoate meglumine) (Winthrop), Reno-M-DIP® (amidotrizoate meglumine; diatrizoate meglumine) (Squibb), Urovist® Meglumine DIU/CT (diatrizoate meglumine) (Berlex), Hypaque® Sodium 25% (diatrizoate sodium) (Winthrop), Conray® 43 (iothalamate meglumine) (Mallinckrodt), Angiovist® 282 (diatrizoate meglumine) (Berlex), Hypaque® Meglumine 60% (diatrizoate meglumine) (Winthrop), Reno-M-60® (diatrizoate meglumine) (Squibb), Conray® (iothalamate meglumine) (Mallinckrodt), Angiovist® 292 (diatrizoate meglumine sodium) (Berlex), MD-60® (diatrizoate meglumine sodium) (Mallinckrodt), Renografin®-60 (diatrizoate meglumine sodium) (Squibb), Hypaque® Sodium 50% (diatrizoate sodium) (Winthrop), Renovue®-65 (iodamide meglumine) (Squibb), Urovist® Sodium 300 (diatrizoate sodium) (Berlex), Renovist®II (diatrizoate meglumine sodium) (Squibb), Hexabrix (ioxaglate meglumine sodium) (Mallinckrodt), Conray® 325 (iothalamate sodium) (Mallinckrodt), Diatrizoate Meglumine 76% (diatrizoate meglumine) (Squibb), Angiovist® 370 (diatrizoate meglumine sodium) (Berlex), Hypaque®-76 (diatrizoate meglumine sodium) (Winthrop), MD-76® (diatrizoate meglumine sodium) (Mallinckrodt), Renografin®-76 (diatrizoate meglumine sodium) (Squibb), Renovist® (diatrizoate meglumine sodium) (Squibb), Hypaque®-M, 75% (diatrizoate meglumine sodium) (Winthrop), Conray® 400 (iothalamate sodium) (Mallinckrodt), Vascoray® (iothalamate meglumine sodium) (Mallinckrodt), Hypaque®-M, 90% (diatrizoate meglumine sodium) (Winthrop) and Angio Conray® (iothalamate sodium) (Mallinckrodt).

Non-ionic contrast agents suitable for use in the methods of the invention include: Isovue®-128 (Squibb) (iopamidol 26.1%), Omnipaque® 140 (Winthrop) (iohexol 30.2%), Optiray® 160 (Mallinckrodt) (ioversol 33.9%), Isovue®-200 (Squibb) (iopamidol 40.8%), Omnipaque® 240 (Winthrop) (iohexol 51.8%), Optiray® 240 (Mallinckrodt) (ioversol 50.9%), Isovue®-300 (Squibb) (iopamidol 61.2%), Omnipaque®-300 (Winthrop) (iohexol 64.7%), Optiray® 320 (Mallinckrodt) (ioversol 67.8%), Omnipaque® 350 (iohexol 75.5%) (Winthrop), Isovue®-370 (Squibb) (iopamidol 75.5%) and iodixanol.

Contrast agents can be administered to the human by, for example, parenteral injection (e.g., intravenously, intra-arterially, intra-thecally, intra-abdominally, subcutaneously, intramuscularly), orally (e.g., as a tablet or a drink) or as an enema. In a particular embodiment, the injection of the contrast agent is a parenteral injection.

“Undergoing,” as used herein, in reference to injection of a contrast agent, means any time before (also referred to herein as prior to), during (also referred to herein as essentially simultaneously) or following (also referred to herein as “after”) administration of the contrast agent to the human. For example, a human can be administered an iron chelator before (e.g., days, hours, minutes) the administration (e.g., injection of the contrast agent). Alternatively or additionally, the human can be administered the iron chelator essentially simultaneously with administration of the contrast agent. Likewise, the human can be administered the iron chelator after the administration of the contrast agent, for example, minutes, hours or days following the administration of the contrast agent. In another embodiment, the human can be administered the iron chelator before, during and after administration of the contrast agent.

In one embodiment, the injection of a contrast agent occurs during a cardiac procedure (e.g., a cardiac catheterization). “Cardiac procedure,” as used herein, refers to any technique involving the heart and blood vessel (artery, vein) associated with the heart.

In another embodiment, the injection of a contrast agent occurs during a non-cardiac procedure. “Non-cardiac procedure,” as used herein, refers to any technique that involves any organ (e.g., intestine, kidney, brain), region of the body (e.g., thorax, head, pelvis) or blood vessel that is not a cardiac procedure.

“Occurs during,” as used herein, means that the cardiac or non-cardiac procedure takes place essentially simultaneously or in relatively immediate succession to administration (e.g., injection) of the contrast agent. For example, a cardiac catheterization includes the placement of a catheter into a blood vessel and then into the heart. A contrast agent is injected through the catheter to assess whether narrowing or blockages are present in the coronary arteries and to assess how the heart valves and heart muscle function. Thus, injection of the contrast agent occurs during the cardiac procedure.

The non-cardiac procedure can be an angiogram. The angiogram can be an angiogram of a blood vessel of at least one member selected from the group consisting of an aorta, a carotid blood vessel, an iliac blood vessel, a femoral blood vessel, a mesenteric blood vessel and a cerebral blood vessel. The blood vessel can be an artery or a vein. The angiogram can also be a venogram.

The angiogram can be an angiogram of at least one member of a region of the body of the human selected from the group consisting of a head region, a thoracic region, an abdominal region, a pelvic region (e.g., pyelogram), an upper extremity region (e.g., hand, forearm, arm) and a lower extremity (e.g., foot, leg, thigh). For example, an angiogram of the head region can include an angiogram of the brain and vessels (blood vessels and lymphatic vessels) to and from the brain; an angiogram to the thoracic region can include an angiogram of the lungs, heart, esophagus and vessels associated with the lungs, heart and esophagus; an angiogram of the abdominal region can include the stomach, small intestine, liver, pancreas, spleen and associated vessels; and an angiogram of the pelvic region can include the reproductive organs, kidneys, ureter and associated blood vessels.

In yet another embodiment, the non-cardiac procedure is a fistulagram (e.g., a dialysis shunt fistulagram).

An “iron chelator,” as used herein, refers to any molecule capable of interacting with iron, either Fe³⁺ or Fe²⁺, to prevent the formation of catalytic iron from Fe³⁺ or to prevent, inhibit or interfere with iron (Fe³⁺ or Fe²⁺) interacting, effecting or participating in the Haber-Weiss reaction or any other reaction which can generate hydroxyl radicals. The interaction between the iron chelator and iron, either Fe³⁺, Fe²⁺, or both, can be, for example, a binding interaction, an interaction as a result of steric hindrance or any reciprocal effect between iron and the iron chelator. The iron chelator can, for example, prevent the conversion of Fe³⁺ to Fe²⁺, thereby indirectly preventing the reduction of hydrogen peroxide and formation of hydroxyl radicals in the Haber-Weiss reaction. Alternatively, or additionally, the iron chelator can interact directly with Fe²⁺ to prevent hydroxyl radical formation in, for example, the Haber-Weiss reaction.

The iron chelator can be a peptide comprising natural or nonnatural (e.g., amino acids not found in nature) amino acids, polyethylene glycol carbamates, lipophilic or nonlipophilic polyaminocarboxylic acids, polyanionic amines or substituted polyaza compounds. In a preferred embodiment, the iron chelator is deferiprone (1,2-dimethyl-3-hydroxy-pyrid-4-one)L1. Iron chelators are commercially available or can be synthesized or purified from biological sources using routine procedures. Exemplary descriptions and discussions of iron chelators are found in several references, for example, U.S. Pat. No. 5,047,421 (1991); U.S. Pat. No. 5,424,057 (1995); U.S. Pat. No. 5,721,209 (1998); U.S. Pat. No. 5,811,127 (1998); Olivieri, N. F. et al., New Eng. J. Med. 332:918-922 (1995); Boyce, N. W. et al., Kidney International. 30:813-817 (1986); Kontoghiorghes, G. J. Indian J. Peditr. 60:485-507 (1993); Hershko, C. et al., Brit. J. Haematology 101:399-406 (1998); Lowther, N. et al., Pharmac. Res. 16:434 (1999), the teachings of all of which are hereby incorporated by reference in their entirety.

An “amount effective,” when referring to the amount of iron chelator, means that amount (also referred to herein as “dose”) of iron chelator that, when administered to a human, is sufficient for therapeutic efficacy (e.g., an amount sufficient to prevent the onset of acute renal failure, to reduce the severity of acute renal failure or to prevent irreversible kidney damage, such as to reduce blood urea nitrogen or creatinine in a blood sample obtained from a human; or to increase GFR in the human). An effective amount of an iron chelator also refers to an amount of iron chelator that when administered to the human prevents a further or an additional increase in a parameter indicative of kidney damage (e.g., an increase in urinary catalytic iron content, an increase in blood urea nitrogen, a decrease in GFR) in a human undergoing injection of a contrast agent compared to the parameter prior to treatment or during treatment with the iron chelator or compared to a control (also referred to herein as “normal”) human or human without a kidney disease or a human not undergoing injection of a contrast agent. Such parameters can be measured in urine or serum of humans undergoing treatment with iron chelator before, during or after the administration of the iron chelator.

In one embodiment, the iron chelator is administered in a single dose. In another embodiment, the iron chelator is administered in multiple doses. The iron chelator can be administered orally at a dose in a range between about 20 mg/kg body of the human and about 150 mg/kg body weight of the human. The iron chelator can be administered several times a day (e.g., three times a day) at a dose in a range of between about 20 mg/kg body of the human and about 150 mg/kg body weight of the human for days, weeks or months before and after injection of the contrast agent.

Iron chelators and parameters associated with kidney diseases (e.g., serum and urinary creatinine, blood urea nitrogen), are also described in U.S. Pat. Nos. 6,906,052, and 6,908,733; and U.S. Patent Application Nos. 20040192663 and 20050026814, the teachings of all of which are hereby incorporated by reference in their entirety.

Administration of an iron chelator to human with or without undergoing an injection of a contrast agent can further include measuring catalytic iron content in a urine sample obtained from a human, as described for example, in U.S. Pat. Nos. 6,906,052 and 6,908,733 and U.S. Patent Application Nos. 20050026814 and 20040192663.

“Catalytic iron” refers to Fe²⁺. Catalytic iron is capable of catalyzing free radical reactions. Iron in the Fe²⁺ state can catalyze the Haber-Weiss reaction, which reduces hydrogen peroxide and promotes formation of hydroxyl radicals. The Haber-Weiss reaction is illustrated below:
O₂⁻+Fe³⁺→O₂+Fe²⁺
Fe²⁺+H₂O₂→Fe³⁺+OH.+OH^{−}
O₂⁻+H₂O₂→O₂+OH.+OH⁻

It is also envisioned that catalytic iron can result in the formation of hydroxyl radicals in reactions other than the Haber-Weiss reaction.

Methods to determine the catalytic iron content are known (see, for example, Gutteridge, J. M. C. et al., Biochem J. 199:263-265 (1981); Yergey, A. L. J. Nutrition 126:355S-361S (1996); Iancu, T. C. et al., Biometals 9:57-65 (1996); Artiss, J. D. et al., Clin. Biochem. 14:311-315 (1981); Smith, F. E. et al., Clin. Biochem. 17:306-310 (1984); Artiss, J. D., et al., Microchem. J. 28:275-284 (1983), the teachings of all of which are hereby incorporated by reference in their entirety) and as described in the exemplification. The methods include spectrophotometric, mass spectrometry (e.g., thermal ionization mass spectrometry, laser microprobe mass analysis, inductively coupled plasma mass spectrometry, atom bombardment-secondary ion mass spectrometry). Catalytic iron content is also referred to as an amount, level or concentration of catalytic iron.

The catalytic iron content can be measured in relation to a reference protein in the urine sample. The phrase “in relation to a reference protein,” as used herein, refers to expressing catalytic iron content in urine as a unit of measurement (e.g., nanomoles) per a concentration of a protein (e.g., milligrams) in the urine. Catalytic iron can be expressed, for example, as nanomoles (nmoles) of catalytic iron per milligram (mg) of protein in urine. The reference protein can be urinary creatinine.

Catalytic iron content in the urine can also be measured in relation to Glomerular Filtration Rate (GFR), in particular, creatinine clearance. GFR is an indicia of renal function which generally refers to the renal (also referred to herein as kidney) excretory capacity. Indices to assess glomerular filtration include, for example, creatinine clearance and inulin clearance. Exemplary descriptions and discussions of techniques to assess renal function, including glomerular filtration rate (e.g., creatinine clearance) are found, for example, in Larsen, K. Clin. Chem. Acta. 41:209-217 (1972); Talke, H. et al., Klin, Wscr 41:174 (1965); Fujita, Y. et al., Bunseki Kgaku 32:379-386 (1983); Rolin, H. A. III, et al., In: “The Principles and Practice of Nephrology”, 2nd ed., Jacobson, H. R., et al., Mosby-Year Book, Inc., St. Louis, Mo., page 8-13 (1995); Carlson, J. A., et al., In: “Diseases of the Kidney”, 5th Ed., Schrier, R. W. et al., eds., Little, Brown and Co., Inc., Boston, Mass., pages 361-405 (1993), the teachings of all of which are hereby incorporated by reference in their entirety. For example, endogenous creatinine clearance can be determined as follows:
Cr=Ucr V/PCr
where Cr=clearance of creatinine (ml/min); Ucr=urine creatinine (mg/dl), V=volume of urine (ml/min−for 24-hr volume: divide by 1400, and PCr=plasma creatinine (mg/dl).

In another embodiment, creatinine, blood urea nitrogen, or both are measured in a blood sample obtained from the human at one or more time points before, during or after the administration of the iron chelator to a human undergoing injection of the contrast agent. The blood sample can be an arterial blood sample or venous blood sample. The blood sample can be a serum or plasma blood sample. Methods to obtain blood samples and TO process the blood sample to determine blood urea nitrogen content and creatinine content are well known to one of skill in the art. (See, for example, Karlinsky, M. L. et al., Kidney Int. 17:293-302 (1980); Tomford, R. C. et al., J. Clin. Invest. 68:655-664 (1981); Baliga, R. et al., Biochem J. 291:901-905 (1993), the teachings of all of which are incorporated herein by reference in their entirety).

In another embodiment, cystatine C content can also be measured in the blood sample obtained from the human undergoing an injection of a contrast agent. Techniques to measure cystatine C in blood samples are known to one of skill in the art (see, for example, Massey, D., J. Clin. Lab. Analysis 18:55-60 (2004); Laterza, O. F., et al., Clin Chem. 48:699-707 (2002); Delanaye, P., et al., Intensive Care Med. 30:980-983 (2004); and Herget-Rosenthal, S., et al., Ann Clin Biochem. 41:111-118 (2004)).

Serum creatinine, blood urea nitrogen content, cystatin C content in a blood sample from the human can increase and GFR can decrease in a human undergoing injection of a contrast agent. An increase in serum creatinine, blood urea nitrogen and cystatin C; and a decrease in GFR be indicative of acute renal failure in the human undergoing injection of a contrast agent. In addition, functional and structural changes associated with acute renal failure consequent to injection of a contrast agent, include, for example, renal vasoconstriction, diuresis, natriuresis, enzymuria and cytoplasmic vacuolization. Administration of the iron chelator to the human undergoing injection of a contrast agent can essentially prevent the onset of an acute renal failure in the human.

“Essentially prevents the onset of acute renal failure,” as used herein, means that acute renal failure in the human is effectively stopped or prevented from occurring. Indices to determine whether administration of the iron chelator essentially prevents the onset of acute renal failure include, for example, assessment of parameters associated acute renal failure, such as a decrease in GFR, an increase blood urea nitrogen, serum creatinine and/or a serum cystatin C compared to a control level (e.g., a human without acute renal failure, a human not undergoing an injection of a contrast agent or a level observed before injection of the contrast agent).

The human treated with the iron chelator while undergoing the injection of a contrast agent, can have acute renal failure consequent to injection of the contrast agent. “Consequent to injection of the contrast agent,” as used herein, means that the contrast agent induces functional and structural changes in the kidney associated with kidney damage or disease (e.g., acute renal failure). Exemplary changes include, for example, renal vasoconstriction, decreased GFR, natriuresis, enzymuria, cytoplasmic vacuolization, retention of nitrogenous waste products in the blood as measured by an increase in blood urea nitrogen or serum creatinine (e.g., greater than about 0.3 mg/dL) or an increase in cystatin C (e.g., at least about 25% increase) compared to a control level or a level observed before injection of the contrast agent.

Administration of the iron chelator to a human undergoing an injection of a contrast agent can reduce the severity of acute renal failure consequent to the injection of the contrast agent. “Reduce the severity,” as used herein when referring to acute renal failure in the human, means any diminution, amelioration or decrease in damage to the kidney (e.g., structural or functional) that compromises the function of the kidney and is associated with acute renal failure (e.g., decrease GFR, retention of nitrogenous waste products in the blood, such as an increase in blood urea nitrogen). The diminution, amelioration or decrease in kidney damage in a human following an administration in the iron chelator can be compared to a parameter from a human prior to administering the iron chelator or to a control. Reducing the severity of the acute renal failure in the human can result, for example, from an amelioration of a primary pathology of the kidney (e.g., injury to the glomerulus or tubule) or another organ which adversely effects the ability of the kidney to perform biological functions in (e.g., eliminate nitrogenous waste products, such as blood urea nitrogen). Thus, reducing the severity of acute renal failure in the human can be a direct or indirect effect of a reduction in the acute renal failure in the human. A reduction in the severity of acute renal failure in the human can be a reduction in kidney damage consequent to the acute renal failure as assessed, for example, by renal vasoconstriction diuresis, increased GFR, decreased serum creatinine or decreased cystatin C.

The administration of the iron chelator to the human with or without undergoing an injection of a contrast agent can prevent irreversible kidney damage consequent to injection of the contrast agent. “Irreversible kidney damage,” as used herein, means that the kidney suffers permanent structural or functional loss. Consequent to injection to the contrast agent, the kidney may be damaged in a manner that affects kidney function and be clinically observed by parameters characteristic of kidney damage, for example, a decreased GFR and increased blood urea nitrogen. Administration of the iron chelator to the human undergoing an injection of a contrast agent can prevent irreversible kidney damage consequent to injection of the contrast agent. For example, a human undergoing injection of a contrast agent may have a decrease in GFR. Following administration of the iron chelator to the human, the GFR may increase or a decrease in GFR may be prevented. Prevention of irreversible kidney damage can be assessed by an increase in GFR that approaches a control value (e.g., the value observed in a human without kidney damage) or an increase to a value above that observed when the kidney is damaged.

Acute renal failure consequent to injection of the contrast agent or the severity of acute renal failure in a human who is healthy and with normal kidneys (e.g., disease-free) is uncommon. Irreversible kidney damage in a healthy human consequent to injection of a contrast agent is infrequent. However, in humans with reduced renal function or conditions causing reduction of renal perfusion, injection of a contrast agent can lead to the development of nephrotoxicity (i.e., contrast nephrotoxicity). Risk factors for developing acute renal failure consequent to injection of a contrast dye include, for example, age, diabetes mellitus, chronic renal insufficiency, congestive heart failure and reduced renal perfusion (see for example, Eisenberg, R. L., et al., Am. J. Roentgenology, 136: 859-861 (1981); Lautin, E. M., et al., Am. J. Roentgenol. 157: 59-65 (1995); Barrett, B. J., J. Am. Soc. Nephrol. 2: 125-137 (1994), the teachings of all of which are hereby incorporated by reference in their entirety).

Although animal models have been used to study some kidney diseases, animal models suffer from a number of limitations when extended to human kidney disease diagnosis and treatment. For example, animal models of kidney disease do not necessarily depict kidney disease in humans, and many treatments of kidney disease in experimental animal models are relatively ineffective in the treatment of acute renal failure in humans (Siegel, N. J. et al., Kidney Int. 25:906-911 (1984); Cronin, R. E., et al., Am. J. Physiol. 248:F332-F339 (1985); Cronin, R. E., et al, Am. J. Physiol. 251: F408-F416 (1986); Seiken, G. et al., Kidney Int. 45:1622-1627 (1994); Shaw, S. G. et al., J. Clin. Invest. 80:1232-1237 (1987); Allgren, R. L. et al., N. Eng J. Med. 336:828-834 (1997); Paller, M. S., Sem. Nephrol. 18:482-489 (1998) ). A treatment found to be effective in animals (Hanns, et al J Am Soc Nephrol 1:612, 1990) cannot necessarily expected to be effective in humans.

There are currently no available effective treatments for acute renal failure, including acute renal failure consequent to injection of a contrast agent. Attempts to treat acute renal failure with treatment options such as the administration dopmamine, diuretics, calcium channel antagonists, aminophylline, atrial natriuretic peptide, fenoldopam and angiotensin-converting enzyme inhibitors do not prevent or reduce the severity of acute renal failure, including acute renal failure consequent to injection of a contrast agent (Solomon R., et al., New Eng J Med 33:1416-1420 (1994); Erickson C. W., et al., Am J. Kid Di 39:A16, (2002); Stevens M. A., J Am College Cardiol, 33:402-411 (1999); Chu V. L., Ann Pharmacother 35:1278-1282 (2001); Early C. M., et al., Kidney Int 45:1425-1431 (1994); Durham J. D., et al., Kidney Int 62:2202-2207, (2002); Kurnick B R, et al., Am J Kid Dis 31:674-680 (1998)) or prevent irreversible kidney damage consequent to injection of contrast agent, prevent mortality and the cost of care associated with acute renal failure (Levy E M, et al., J Am Med Assoc, 275:1516-1517 (1996); Gruberg L, et al., J Am College Cardiol 36:1542-1548 (2000), Riha I. C. S., et al., Circulation 105: 2259-64 (2002)). Recently acetylcysteine has been administered to humans with acute renal failure with conflicting results (Misrad, et al., Clinical Card, 27.-11:607-610 (2004); Alonso, et al., Am J Kidney Dis 43:1 (2004); Birk, et al., Lancet 362:598-603 (2003); Isanbarger et al., Am J of Card 92:1454-1458 (2003); Kshisager et al, J Am Soc Nephrol, 15:761-769 (2004); Pannu et al, KI, 65:1366 (2004) including no effect observed (Nallamothu et al., Am J Med 117:938-947 (2005).

Acute renal failure, in particular, CN, can include an impairment in renal function, for example, an increase in serum creatinine of greater than 25% or 0.3 mg/dL (44 micro mol/L from baseline) within three days after injection of the contrast agent. CN is a significant cause of hospital-acquired renal failure (Morcos, S. K., J. Vasc. Interv. Radiol. 16: 13-23 (2005)). Humans at high risk for developing acute renal. failure, for example CN, are humans with preexisting renal impairment (serum creatinine levels greater than 1.5 mg/dL (1.30 mol/L)), particularly when the reduction in renal function is associated with diabetes mellitus (Solomon, R., Kidney Int. 53: 230-242 (1998); Morcos, S. K., et al., Eur. Radiol. 9; 1602-1613 (1999)). A “pre-existing renal impairment,” as used herein, means a reduction in a functional or structural property of the kidney, such as assessed by, for example, GFR, creatinine clearance, blood urea nitrogen, serum or plasma creatinine, serum cystatin C, or any combination thereof.

The preexisting renal impairment can be a progressive kidney disease. A progressive kidney disease treated by the methods described herein, includes any kidney disease that can, ultimately, lead to end-stage renal disease. The progressive kidney disease can be can be a glomerular kidney disease, for example, diabetic nephropathy (e.g., as a consequence of Type I or Type II diabetes or systemic lupus), primary glomerulonephritis (e.g., membranous nephropathy, focal segmental glomerulosclerosis, membranoproliferative glomerulonephritis, diffuse proliferative glomerulonephritis, membranous focal segmental glomerulosclerosis) and secondary glomerulonephritis (e.g., diabetic nephropathy, ischemic nephropathy).

The progressive kidney disease can include a chronic kidney disease. Chronic kidney disease is a gradual and progressive loss of the ability of the kidneys to excrete waste, concentrate urine and conserve electrolytes. Chronic kidney disease is also referred to as chronic renal insufficiency and chronic kidney failure. Unlike acute renal failure with its sudden reversible failure of kidney function, chronic renal failure is slowly progressive. It can result from any disease that causes gradual loss of kidney function and can range from mild dysfunction of the kidney to severe kidney failure. Progression of the chronic kidney disease can continue to end-stage renal disease. Chronic kidney disease usually occurs over a number of years as the internal structures of the kidney are slowly damaged. Clinical parameters indicative of chronic kidney disease include an increase in creatinine levels, an increase in blood urea nitrogen levels, a decrease in creatinine clearance and a decrease in GFR. The human having a progressive kidney disease (e.g., chronic kidney disease) undergoing an injection of a contrast agent can have acute renal failure consequent to injection of the contrast agent. Administration of the iron chelator to the human having a progressive kidney disease and acute renal failure consequent to injection of the contrast agent can reduce the severity of the acute renal failure.

Administration of the iron chelator to a human undergoing injection of a contrast agent (e.g., to a human that has a preexisting renal impairment such as chronic kidney disease) can lower blood urea nitrogen or creatinine content in a serum sample of a human; or increase GFR to about that of a control level. “Control level,” as used herein, means a level of a parameter of interest (e.g., blood urea nitrogen or creatinine content in a blood sample or in urine; or GFR) in a human not undergoing injection of a contrast agent and/or a human undergoing injection of a contrast agent, but not having a preexisting renal impairment, matched as necessary for variables, such as age, sex, ethnicity and health history, with a human being treated with the iron chelator while undergoing an injection of a contrast agent. A control level can also be the expected level of a parameter of interest in a human. The “expected level,” as used herein, of a parameter of interest in the human treated by the methods of the invention can be an amount normally observed in a human not undergoing injection of a contrast agent and/or not having a preexisting renal impairment (e.g., progressive kidney disease) or acute renal failure. An expected level can also be any level of a parameter of interest that is below the level prior to treatment with the iron chelator or lower than a level expected as a consequence of acute renal failure or associated with chronic kidney disease in a human not undergoing injection of a contrast agent.

Administration of an iron chelator to a human undergoing an injection of a contrast agent can prevent morbidity or mortality associated with acute renal failure.

The methods of the present invention can be accomplished by the administration of the iron chelator iron by enteral or parenteral means. Specifically, one route of administration is by oral ingestion (e.g., tablet, capsule form). The iron chelator can be administered in an immediate release tablet, a delayed release tablet and/or a extended/slow release tablet to a human with or without the human undergoing an injection of a contrast agent. Other routes of administration can also encompassed by the present invention including intramuscular, intravenous, intraarterial, intraperitoneal, or subcutaneous routes, and nasal administration. Suppositories or transdermal patches can also be employed.

The iron chelators can be administered in an immediate release tablet, a delayed release tablet and/or an extended/slow release tablet to a human with or without the human undergoing an injection of a contrast agent. An “immediate release,” of an iron chelator as used herein, means that the iron chelator is released from the route of administration (e.g., a tablet) within about 1 hour after administration to the human. “Extended release,” of an iron chelator as used herein, means that the iron chelator is released from the route of administration (e.g., a tablet) between about 1 hours and about 3 hours after administration to the human. Extended release is also referred to herein as “slow release.” A “delayed release,” of an iron chelator as used herein, means that the iron chelator is released from the route of administration (e.g., a tablet) at least about 3 hours after administration to the human. Delayed release is also referred to herein as “enteric release.”

The human administered the immediate release tablet, a delayed release tablet and/or an extended/slow release tablet of the iron chelator can have a kidney disease. The kidney disease can be a progressive kidney disease or a renal insufficiency. Renal insufficiency, also referred to as renal failure, occurs when the kidneys no longer have enough kidney function to maintain a normal state of health. Acute renal failure and chronic renal insufficiency are two kinds of renal insufficiency. “Chronic renal insufficiency (CRI),” as used herein, means the kidneys slowly lose their ability to function normally, such as the ability to excrete wastes, concentrate urine and conserve salt and water. The kidney's role in growth can also be compromised, which can lead to growth failure.

The iron chelators can be used alone or in any combination when administered to the humans. For example, deferiprone can be coadministered with another iron chelator such as deferoxiamine to treat a human undergoing an injection of a contrast agent. It is also envisioned that one or more iron chelators can be coadministered with other therapeutics (e.g., dopamine, diuretics, calcium-channel antagonists, aminophylline, atrial natriuretic peptide, insulin-derived growth factor, acetylcysteine and hydration) to, for example, treat acute renal failure in the human consequent to injection of the contrast agent, to prevent the onset of acute renal failure in a human undergoing an injection of a contrast agent, to reduce the severity of acute renal failure in a human consequent to an injection of a contrast agent and/or to prevent irreversible kidney damage in a human consequent to the injection of a contrast agent. Coadministration is meant to include simultaneous or sequential administration of two or more iron chelators. It is also envisioned that multiple routes of administration (e.g., intramuscular, oral, transdermal) can be used to administer one or more iron chelators.

The iron chelators can be administered alone or as admixtures with conventional excipients, for example, pharmaceutically, or physiologically, acceptable organic, or inorganic carrier substances suitable for enteral or parenteral application which do not deleteriously react with the iron chelator. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the iron chelator.

When parenteral application is needed or desired, particularly suitable admixtures for the iron chelator are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The iron chelators can also be administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of both of which are hereby incorporated by reference.

The dosage and frequency (single or multiple doses) of iron chelators administered to a human can vary depending upon a variety of factors, including the size, age, sex, health, body weight, body mass index, and diet of the human; nature and extent of symptoms of the acute renal failure consequent to injection of a contrast agent or the kidney disease and damage to the kidney consequent to injection of the contrast agent, kind of concurrent treatment (e.g., dopamine, diuretics, calcium-channel antagonists, aminophylline, atrial natriuretic peptide, insulin-derived growth factor, acetylcysteine and hydration), complications from the injection of the contrast agent, acute renal failure consequent to the injection of the contrast agent, preexisting renal impairment in the human or other health-related problems. For example, the humans can be treated three times a day with a dose of iron chelator (e.g., deferiprone in 500 mg capsules) at about 30 mg/kg to about 75 mg/kg body weight per day for about 2-6 months. Other therapeutic regimens or agents can be used in conjunction with the iron chelator treatment methods of the present invention. For example, the administration of the iron chelator can be accompanied by administration of dopamine, diuretics, calcium-channel antagonists, aminophylline, atrial natriuretic peptide, insulin-derived growth factor, acetylcysteine and/or hydration. Adjustment and manipulation of established dosages (e.g., frequency and duration) are well within the ability of those skilled in the art.

The present invention is further illustrated by the following examples, which are not intended to be limiting in any way.

### EXEMPLIFICATION

Pharmacokinetics of Deferiprone

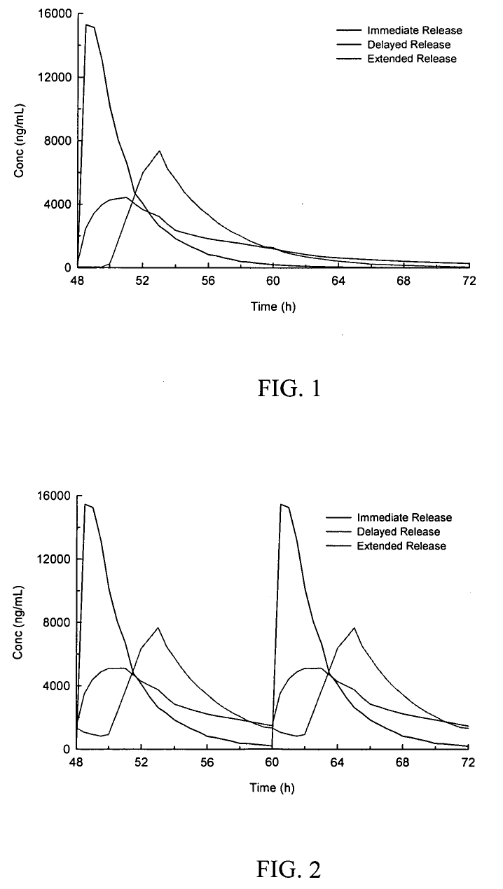

An open-label, single dose, randomized, three-way, crossover study was conducted wherein twelve subjects with mild renal insufficiency (creatinine clearance of 50-80 ml/min) received a single oral dose of 1800 mg (two 900 mg tablets) deferiprone in each of the three study periods following an overnight fast. The three formulations evaluated were 900-mg immediate-release tablets (Treatment A), 900-mg enteric-coated tablets (also referred to herein as “delayed release”) (Treatment B) and 900-mg sustained/extended-release tablets (Treatment C). Blood samples were collected immediately prior to and for a period of 48 hours after each dose and the concentrations of deferiprone determined. The pharmacokinetic parameters which were calculated from the plasma concentration measurements are area under the curve (AUC), maximum concentration (Cmax), time of maximum concentration (Tmax), apparent volume of distribution (Vdss/F) and terminal elimination half-life as shown in the following Table and FIGS. 1 and 2.

TABLE
Pharmacokinetics of Deferiprone

| | AUC | Cmax | Tmax | T½ |
|---|---|---|---|---|
| Treatment A | 43,391 | 15,423 | 1 hr | 2 hrs |
| Treatment B | 36,663 | 6,423 | 5 hrs | 2 hrs |
| Treatment C | 31,558 | 3,280 | 2 hrs | 6 hrs |

The pharmacokinetic profile of deferiprone in patients with mild to moderate renal insufficiency is very similar to that reported with patients with normal renal function. This indicates that deferiprone can be used in these patients. The comparisons of the pharmacokinetic data among immediate-release, delayed-(enterically-coated), and extended/slow-release preparations indicate that the slow-release and enterically-coated preparations may be used twice per day instead of as opposed to three times per day for immediate release. Previous studies with deferiprone have been conducted in patients with iron overload states. These data indicate similar pharmacokinetic profiles in patients with normal iron stores.

### EQUIVALENTS

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A method of treating a human, comprising the step of administering an iron chelator to a human undergoing an injection of a contrast agent.

2. The method of claim 1, wherein the contrast agent is an ionic contrast agent.

3. The method of claim 1, wherein the contrast agent is a non-ionic contrast agent.

4. The method of claim 1, wherein the injection is a parenteral injection.

5. The method of claim 4, wherein the parenteral injection is an intra-arterial injection.

6. The method of claim 4, wherein the parenteral injection is an intravenous injection.

7. The method of claim 1, wherein the injection occurs during a cardiac procedure.

8. The method of claim 7, wherein the cardiac procedure is a cardiac catheterization.

9. The method of claim 1, wherein the injection occurs during an non-cardiac procedure.

10. The method of claim 9, wherein the non-cardiac procedure is an angiogram.

11. The method of claim 10, wherein the angiogram is an angiogram of a blood vessel.

12. The method of claim 11, wherein the blood vessel is at least one member selected from the group consisting of an aorta, a carotid blood vessel, an iliac blood vessel, a femoral blood vessel, a mesenteric blood vessel and a cerebral blood vessel.

13. The method of claim 11, wherein the blood vessel is an artery.

14. The method of claim 11, wherein the blood vessel is a vein.

15. The method of claim 10, wherein the angiogram is of at least one member of a region of the body of the human selected from the group consisting of a head region, a thoracic region, an abdominal region, a pelvic region, an upper extremity region and a lower extremity region.

16. The method of claim 9, wherein the non-cardiac procedure is a fistulagram.

17. The method of claim 16, wherein the fistulagram is a dialysis shunt fistulagram.

18. The method of claim 1, wherein the iron chelator is selected from the group consisting of deferiprone, deferoxamine, polyanionic amines and substituted polyaza compounds.

19. The method of claim 1, wherein the iron chelator is administered at a dose in a range of between about 20 mg/kg body weight and about 150 mg/kg body weight of the human per day.

20. The method of claim 1, wherein the iron chelator is administered in multiple doses.

21. The method of claim 1, wherein the iron chelator is administered orally.

22. The method of claim 1, further including measuring catalytic iron content in a urine sample obtained from the human.

23. The method of claim 22, wherein the catalytic iron is measured in relation to a reference protein in the urine sample.

24. The method of claim 23, wherein the reference protein is urinary creatinine.

25. The method of claim 1, further including measuring creatinine in a blood sample obtained from the human.

26. The method of claim 1, further including measuring blood urea nitrogen content in a blood sample obtained from the human.

27. The method of claim 1, further including measuring cystatin C content in a blood sample obtained from the human.

28. The method of claim 1, wherein administration of the iron chelator essentially prevents the onset of an acute renal failure in the human.

29. The method of claim 1, wherein the human has acute renal failure consequent to the injection of the contrast agent.

30. The method of claim 29, wherein administration of the iron chelator reduces the severity of the acute renal failure in the human.

31. The method of claim 1, wherein the human is administered the iron chelator before the injection of the contrast agent.

32. The method of claim 1, wherein the human is administered the iron chelator during the injection of the contrast agent.

33. The method of claim 1, wherein the human is administered the iron chelator after the injection of the contrast agent.

34. The method of claim 1, wherein administration of the iron chelator prevents irreversible kidney damage in the human consequent to the injection of the contrast agent.

35. The method of claim 1, wherein the human has a preexisting renal impairment.

36. The method of claim 35, wherein the preexisting renal impairment is a progressive kidney disease.

37. The method of claim 36, wherein the progressive kidney disease is a chronic kidney disease.

38. The method of claim 37, wherein administration of the iron chelator essentially prevents the onset of an acute renal failure in the human.

39. The method of claim 37, wherein the human has acute renal failure consequent to the injection of the contrast agent.

40. The method of claim 39, wherein administration of the iron chelator reduces the severity of the acute renal failure.
